SEQUENCE LISTING (14 sequences)

SEQ ID NO: 1 - DNA, 36 bp, Artificial Sequence
  FIP primer Mollicutes
tgcgggtccccgtcaattgcctgggtagtacattcg

SEQ ID NO: 2 - DNA, 36 bp, Artificial Sequence
  BIP primer Mollicutes
caagtggtggagcatgtttgtcaagtctaggtaagg

SEQ ID NO: 3 - DNA, 18 bp, Artificial Sequence
  LPF primer Mollicutes
gtttgagtttcattcttg

SEQ ID NO: 4 - DNA, 18 bp, Artificial Sequence
  LPB primer Mollicutes
cttaattcgacggtacac

SEQ ID NO: 5 - DNA, 18 bp, Artificial Sequence
  B3 primer Mollicutes
tgtttccataactttgcc

SEQ ID NO: 6 - DNA, 18 bp, Artificial Sequence
  F3 pimer Mollicutes
gttaacacattaagtatc

SEQ ID NO: 7 - DNA, 40 bp, Artificial Sequence
  FIP primer Human herpesvirus 1
gttgggtggtggaggagacgtccttttggttcttgtcggt

SEQ ID NO: 8 - DNA, 38 bp, Artificial Sequence
  BIP primer Human herpesvirus 1
ggtcgtccctcgcatgaagcggcgtggtaaggctgatg

SEQ ID NO: 9 - DNA, 18 bp, Artificial Sequence
  LPF primer Human herpesvirus 1
ttggtgggaacccccgat

SEQ ID NO: 10 - DNA, 20 bp, Artificial Sequence
  LPB primer Human herpesvirus 1
aacatgacccagaccggcac

SEQ ID NO: 11 - DNA, 18 bp, Artificial Sequence
  B3 primer Human herpesvirus 1
tacttggcatggggggtg

SEQ ID NO: 12 - DNA, 20 bp, Artificial Sequence
  F3 primer Human herpesvirus 1
gccgttgttcccattatccc

SEQ ID NO: 13 - DNA, 1740 bp, Artificial Sequence
  Mycoplasma pneumoniae strain ATCC 15531 16S ribosomal RNA gene and 16S-23S rRNA intergenic spacer
ttaacgctggcggcatgcctaatacatgcaagtcgatcgaaagtagtaatactttagaggcgaacgggtgagtaacacgtatccaatctaccttataatgggggataactagttgaaagactagctaataccgcataagaactttggttcgcatgaatcaaagttgaaaggacctgcaagggttcgttatttgatgagggtgcgccatatcagctagttggtggggtaacggcctaccaaggcaatgacgtgtagctatgctgagaagtagaatagccacaatgggactgagacacggcccatactcctacgggaggcagcagtagggaatttttcacaatgagcgaaagcttgatggagcaatgccgcgtgaacgatgaaggtctttaagattgtaaagttcttttatttgggaagaatgactttagcaggtaatggctagagtttgactgtaccattttgaataagtgacgactaactatgtgccagcagtcgcggtaatacataggtcgcaagcgttatccggatttattgggcgtaaagcaagcgcaggcggattgaaaagtctggtgttaaaggcagctgcttaacagttgtatgcattggaaactattaatctagagtgtggtagggagttttggaatttcatgtggagcggtgaaatgcgtagatatatgaaggaacaccagtggcgaaggcgaaaacttaggccattactgacgcttaggcttgaaagtgtggggagcaaataggattagataccctagtagtccacaccgtaaacgatagatactagctgtcggggcgatcccctcggtagtgaagttaacacattaagtatctcgcctgggtagtacattcgcaagaatgaaactcaaacggaattgacggggacccgcacaagtggtggagcatgttgcttaattcgacggtacacgaaaaaccttacctagacttgacatccttggcaaagttatggaaacataatggaggttaaccgagtgacaggtggtgcatggttgtcgtcagctcgtgtcgtgagatgttgggttaagtcccgcaacgagcgcaacccttatcgttagttacattgtctagcgagactgctaatgcaaattggaggaaggaagggatgacgtcaaatcatcatgccccttatgtctagggctgcaaacgtgctacaatggccaatacaaacagtcgccagcttgtaaaagtgagcaaatctgtaaagttggtctcagttcggattgagggctgcaattcgtcctcatgaagtcggaatcactagtaatcgcgaatcagctatgtcgcggtgaatacgttctcgggtcttgtacacaccgcccgtcaaactatgaaagctggtaatatttaaaaacgtgttgctaaccattaggaagcgcatgtcaaggatagcaccggtgattggagttaagtcgtaacaaggtacccctacgagaacgtgggggtggatcacctcctttctaatggagttttttactttttcttttcatctttaataaagataaatactaaacaaaacatcaaaatccatttatttatcggtggtaaattaaacccaaatccctgtttggtctcacaactaacatatttggtcagattgtatccagttctgaaagaacatttccgcttctttcaaaactgaaaacgacaatctttctagttccaaataaataccaaaggatcaatacaataagttactaagggcttatggtg

SEQ ID NO: 14 - DNA, 1799 bp, Artificial Sequence
  Human herpesvirus 1 isolate E19 US4 and US5 genes complete cds and US6 gene partial cds
tgaccgcccccggggggcggtgctgtttgcgggttggcacaaaaagaccccgacccgcgtctgtggtgtttttggcatcatgtcgccgggcgccatgcgtgccgttgttcccattatcccattccttttggttcttgtcggtgtatcgggggttcccaccaacgtctcctccaccacccaaccccaactcccgaccaccggtcgtccctcgcatgaagcccccaacatgacccagaccggcaccaccgactctcccaccgccatcagccttaccacgcccgaccacacaccccccatgccaagtatcggactggaggaggaggaggaagaggaggagggggccggggatggcgaacatcttgaggggggagatgggacccgtgacaccctaccccagtccccgggtccagccgtcccgttggccggggatgacgagaaggacaaacccaaccgtcccgtagtcccaccccccggtcccaacaactcccccgcgcgccccgagaccagtcgaccgaagacaccccccaccaggatcgggccgctggcaactcgacccacgacccaactcccctcaaaggggcgacccttggttccgacgcctcaacataccccgctgttctcgttcctcactgcctcccccgccctggacaccctcttcgtcatcagcaccgtcatccacaccttatcgtttgtgtgtattgttgcgatggcgacacacctgtgtggcggttggtccagacgcgggcgacgcacacaccctagcgtgcgttacgtgtgcctgccgcccgaacgcgggtagggtatggggagagcctacacgcggaaagcaagaacaataaaggcggcgggatctagttgatatgcgtctctgggtgtttttggggtgtggcgggcgccgggcggtcattggacggggtgcagttaaatacatgcccgggacccatgaagcatgcgcgacttccgggcctcggaacccacccgaaacggccaacggacgtctgagccaggcctggctatccggagaaacaacacacgacttggcgttctgtgtgtcgcgatgtctctgcgcgcagtctggcatctggggcttttgggaagcctcgtgggggctgttcttgccgccacccatctgggacctgcggccaacacaacggaccccttaacgcacgccccagtgtcccctcaccccagccccctggggggctttgccgtccccctcgtagtcggtgggctgtgtgccgtagtcctgggggcggcgtgtctgcttgagctcctgcgtcgtacgtgccgcgggtgggggcgttaccatccctacatggacccagttgtcgtataagatgtcgggtccaaactcccgacaccaccagctggcatggtataaatcaccggtgcgccccccaaaccatgtccggcagggggatgggcacccaacaacaccgggctaaccaggaaatccgtggccccggcccccaacaaagatcgcggtagcccggccgtgtgacactatcgtccataccgaccacaccgacgaatcccctaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcggcaaatatgccttggcggatgcctctctt

(19) United States
(12) Patent Application Publication
(10) Pub. No.: US 2023/0250498 A1
(43) Pub. Date: Aug. 10, 2023

(54) ISOTHERMAL REAL-TIME PCR METHOD FOR DETERMINING PRESENCE OF A PRE-DETERMINED NUCLEIC ACID SEQUENCE IN HUMAN SAMPLES

(71) Applicant: CERTUS MOLECULAR DIAGNOSTICS AG, Bern (CH)

(72) Inventors: Samuel ZÜRCHER, Bern (CH); Alexander LÜTHI, Bern (CH); Lea WEIBEL, Bern (CH)

(21) Appl. No.: 18/009,142

(22) PCT Filed: Jun. 9, 2021
(86) PCT No.: PCT/EP2021/065547

§ 371 Date: Dec. 8, 2022

(30) Foreign Application Priority Data
Jun. 9, 2020 (EP) 20179110.0

(51) Int. Cl. C12Q 1/70; C12Q 1/6888; A61K 45/06

(52) U.S. Cl. CPC C12Q 1/705; C12Q 1/6888; A61K 45/06

(57) ABSTRACT

The present invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether an elongated DNA sequence is present in the sample, wherein presence of the elongated DNA sequence in the sample is indicative of the presence of the pre-determined nucleic acid sequence in the sample, wherein the sample is obtained from a human subject and wherein no F3 primer is used.

### RELATED APPLICATIONS

The instant application is a 35 U.S.C. § 371 filing of International Patent Application No. PCT/EP2021/065547, filed Jun. 9, 2021, which claims priority to European Patent Application No. 20179110.0, filed Jun. 9, 2020, the entire contents of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on Dec. 7, 2022, is named 737571_VSO9-006US_ST25.txt and is 7,543 bytes in size.

The present invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether an elongated DNA sequence is present in the sample, wherein presence of the elongated DNA sequence in the sample is indicative of the presence of the pre-determined nucleic acid sequence in the sample, wherein the sample is obtained from a human subject and wherein no F3 primer is used.

Various methods exist for determining whether nucleic acid sequences are present in samples obtained from human subjects. Most methods rely on nucleic acid molecules hybridisable to a pre-determined sequence, such as for example a sequence expected to be present in a pathogen.

However, existing tests are either highly specific or quickly provide a result which lacks appropriate reliability. Thus, there is a need in the art for a method providing a highly specific and quick result.

The above technical problem is solved by the embodiments provided herein and as characterized in the claims.

Accordingly, the present invention relates to, inter alia, the following embodiments.
1. A method for determining presence of a pre-determined nucleic acid sequence in a sample, the method comprising the steps of:
   (a) adding one or more enzyme(s) providing activities of RNA- and/or DNA-dependent DNA polymerase activity and strand-displacement activity to the sample to be analysed for the presence of the pre-determined nucleic acid sequence;
   (b) adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence;
   (c) incubating the sample resulting from steps (a) and (b) at a fixed temperature;
   (d) determining whether an elongated DNA sequence is present in the sample, wherein presence of the elongated DNA sequence in the sample is indicative of the presence of the pre-determined nucleic acid sequence in the sample
   wherein the sample is obtained from a human subject and wherein no F3 primer is used.
2. The method of embodiment 1, wherein four of the at least five primers are forward inner primer (FIP), backward inner primer (BIP), loop primer forward (LPF) and loop primer backwards (LPB), respectively.
3. The method of embodiment 1 and 2, wherein the fifth primer is a B3 primer.
4. The method of any one of embodiments 1 to 3, wherein the pre-determined nucleic acid sequence is an RNA or DNA sequence.
5. The method of any one of embodiments 1 to 4, wherein the pre-determined RNA or DNA sequence is comprised in a pathogen.
6. The method of embodiment 5, wherein the pathogen is a virus, a bacterium, a fungus or a parasite.
7. The method of embodiment 6, wherein the pathogen is a human herpesvirus or a bacterium of the genus Mycoplasma.
8. The method of any one of embodiments 1 to 7, wherein the fixed temperature is between 50 and 75° C.
9. The method of any one of embodiments 1 to 8, wherein the sample in step (c) is incubated for 1 to 120 minutes.
10. The method of any one of embodiments 1 to 9, wherein presence of the double-stranded elongated DNA sequence in the sample is determined by using a nucleic acid molecule hybridisable to the double-stranded elongated DNA sequence, in particular wherein the nucleic acid molecule is labelled, using a molecule that intercalates in the double-stranded elongated DNA sequence or using turbidity measurement.
11. An anti-infective composition for use in the treatment of an infection of a pathogen, wherein the subject has previously been determined to be infected by the pathogen using the method of any one of embodiments 1 to 10.
12. The anti-infective composition for use of embodiment 11, wherein the pathogen is a virus, a bacterium, a fungus or a parasite.
13. The anti-infective composition for use method of embodiment 11 or 12, wherein the anti-infective composition comprises an antiviral, antibiotic, antifungal or antiparasitic drug, respectively.
14. The anti-infective composition for use of any one of embodiments 12 to 13, wherein the pathogen is a human herpesvirus or a bacterium of the genus Mycoplasma.

Accordingly, in a first aspect, the invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether a double-stranded elongated DNA sequence is present in the sample, wherein presence of the double-stranded elongated DNA sequence in the sample is indicative of the presence of the pre-determined nucleic acid sequence in the sample, wherein the sample is obtained from a human subject and wherein no F3 primer is used.

The term “pre-determined nucleic acid sequence”, as used herein, refers to a nucleic acid sequence, preferably an RNA or DNA sequence, where the skilled person is aware that it may be comprised in a sample obtained from a human subject (e.g. tissue sample, bronchoalveolar lavage, bronchial wash, pharyngeal exudate, tracheal aspirate, blood, serum, plasma, bone, skin, soft tissue, intestinal tract specimen, genital tract specimen, breast milk, lymph, cerebrospinal fluid, pleural fluid, sputum, urine, a nasal secretion, tears, bile, ascites fluid, pus, synovial fluid, vitreous fluid, vaginal secretion, semen and/or urethral tissue). In particular, the pre-determined nucleic acid sequence, within the present invention, is a sequence that is detectable using the method of the present invention. That is, a nucleic acid sequence available to the skilled person is pre-determined if the skilled person can determine whether the sequence will likely be detectable in a sample obtained from a human subject using the methods as provided herein. Within the present invention, the pre-determined nucleic acid sequence comprises at least one primer binding site that is at least partially identical to at least one of the primers used in the methods of the invention. Primer binding sites are considered identical to a primer site if the sequence is exactly identical or if they differ only in that one sequence comprises uracil instead of thymidine and/or if they differ only in that one sequence comprises one or more modified nucleotides instead of the respective non-modified nucleotide(s).

The terms “DNA primer” or “primer”, as used herein, refer to a nucleic acid molecule comprising a 3′-terminal —OH group that, upon hybridisation to a complementary nucleic acid sequence, can be elongated, e.g., via an enzymatic nucleic acid replication reaction. The primer set according to the present invention is used for amplification of nucleic acids, that is, for a LAMP analysis or a RT-LAMP analysis. Both the upper and lower limits of the length of the primer are empirically determined. The primer described herein can be a forward primer or a reverse primer. The term “backward primer”, as used herein, refers to a primer priming the antisense strand of a DNA sequence to allow the polymerase to extend in one direction along the complementary strand of a DNA sequence. At least one backward primer also serves as the RT primer for reverse transcription. The term “forward primer”, as used herein, refers to a primer priming the sense strand of a DNA sequence to allow a polymerase to extend in one direction along one strand of a DNA sequence.

An enzyme providing activities of RNA- and/or DNA-dependent DNA polymerase activity can synthesize DNA in the 5'->3′ direction based on a template composed of a DNA or RNA strand. As the skilled person is aware, such an enzyme will be successively adding nucleotides to the free 3′-hydroxyl group of the template. In this regard, the template strand determines the sequence of the added nucleotides based on Watson-Crick base pairing. The activity of the DNA polymerase may be RNA- and/or DNA-dependent. Exemplary polymerases include, but are not limited to Bst DNA polymerase, Vent DNA polymerase, Vent (exo-) DNA polymerase, Deep Vent DNA polymerase, Deep Vent (exo-) DNA polymerase, Bca (exo-) DNA polymerase, DNA polymerase I Klenow fragment, Φ29 phage DNA polymerase, Z-Taq™ DNA polymerase, ThermoPhi polymerase, 9° Nm DNA polymerase, and KOD DNA polymerase. See, e.g., U.S. Pat. Nos. 5,814,506; 5,210,036; 5,500,363; 5,352,778; and 5,834,285; Nishioka, M., et al. (2001) J. Biotechnol. 88, 141; Takagi, M., et al. (1997) Appl. Environ. Microbiol. 63, 4504.

As an enzyme providing activities of RNA-dependent DNA polymerase activity any suitable reverse transcriptase may be employed. In this regard, the enzyme to be used is not particularly limited, with the proviso that it has the activity to synthesize cDNA using RNA as the template. In addition, a substance which improves heat resistance of the nucleic acid amplification enzyme, such as trehalose, can be added.

When simply expressed as “5′-end side” or “3′-end side” in this specification, it means the direction in the chain which is regarded as the template in all cases. Also, when described that the 3′-end side becomes the starting point of complementary chain synthesis, it means that the 3′-end side —OH group is the starting point of complementary chain synthesis.

The term “strand displacement”, as used herein, refers to the ability of an enzyme to separate the DNA and/or RNA strands in a double-stranded DNA molecule and/or in a double-stranded RNA molecule during primer-initiated synthesis.

The term “hybridisation”, as used herein, refers to the annealing of complementary nucleic acid molecules. When two nucleic acids “hybridise to” each other, or when one nucleic acid “hybridises to” another, the two nucleic acid molecules exhibit a sufficient number of complementary nucleobases that the two nucleic acid molecules can anneal to each other under the particular conditions (e.g., temperature, salt and other buffer conditions) being utilized for a particular reaction. The most common mechanism of hybridisation involves hydrogen bonding (e.g., Watson-Crick, Hoogsteen or reversed Hoogsteen hydrogen bonding) between complementary nucleobases of the nucleic acid molecules. Hybridisation can occur under varying conditions. Stringent conditions are sequence-dependent and are determined by the nature and composition of the nucleic acid molecules to be hybridised. Nucleic acid hybridisation techniques and conditions are known to the skilled artisan and have been described extensively. See, e.g., Sambrook et al, Molecular Cloning: A Laboratory Manual 2nd ed. Cold Spring Harbor Press, 1989; Ausubel et al, 1987, Current Protocols in Molecular Biology; Greene Publishing and Wiley-Interscience, New York; Tijessen, 1993, Hybridization with Nucleic Acid Probes, Elsevier Science Publishers, B.V.; and Kricka, 1992, Non-Isotopic DNA Probe Techniques, Academic Press, San Diego, Calif.

The term “F3”, as used herein, refers to the outer forward primer of a primer set.

Within the present invention, it was surprisingly found that a five-primer system, wherein the F3 primer is omitted, is most efficient in detecting a pre-determined nucleic acid sequence. “Most efficient” as used herein means that detection is as fast and sensitive than commonly used techniques but maintains reliability, which is a prerequisite in tests used for detecting nucleic acids such as nucleic acids derived from a human herpesvirus or a bacterium of the genus Mycoplasma. In addition, it was found that by using five primers instead of six primers as in the standard LAMP technology, shorter target sequences can be detected.

The invention provides a sample containing a pre-determined nucleic acid sequence, and a method for amplifying a nucleic acid, which comprises carrying out an amplification reaction of the pre-determined nucleic acid sequence in the sample, in a reaction system wherein at least one primer of the invention is present. In certain embodiments of the invention, at least one species of the primers is used in the nucleic acid amplification reaction of the invention. That is, the DNA primer described herein may be used in combination with other primers, or two species of the DNA primer described herein may be used.

It is preferred within the methods of the present invention that five DNA primers are used. In some embodiments, the at least two of the primers employed in the invention are loop primers.

The term “loop primer”, as used herein, refers to a DNA primer comprising a sequence that is hybridisable to at least one loop region of an amplification product of the pre-determined RNA sequence. The loop region is formed by the annealing of a strand of an amplification product to itself. Typically, loop primers hybridise to generated DNA sequences and provide an increased number of starting points for the initiation of further DNA elongation processes. The use of loop primer can accelerate the amplification process.

Within the present invention, it is preferred that the at least five primers comprise a forward inner primer (FIP), backward inner primer (BIP), loop primer forward (LPF) and loop primer backwards (LPB), respectively.

The term “FIP” or “forward inner primer”, as used herein, refers to a forward primer that comprises a sequence for strand initiation and a sequence hybridisable to the same FIP-initiated strand.

The term “BIP” or “backward inner primer”, as used herein, refers to a backward primer that comprises a sequence for strand initiation and a sequence hybridisable to the same BIP-initiated strand.

The term “loop primer forward” or “LPF”, as used herein, refers to a loop primer that is a forward primer.

The term “loop primer backwards” or “LPB”, as used herein, refers to a loop primer that is a backwards primer.

Preferably, the at least five primers further comprise a B3 primer.

The term “B3”, as used herein, refers to the outer backward primer of a primer set.

The DNA Primer described herein that specifically binds to a target nucleic acid or its complementary sequence may be at least 10, 15, or 18 nucleotides in length, at least 10, 15, 17, or 18 nucleotides for B3, at least 25, 30, 33, or 36 nucleotides for FIP and BIP, and at least 10, 15, 17, or 18 for LPF and LPB. DNA Primers that specifically bind to a target nucleic acid sequence may have a nucleic acid sequence at least 80% complementarity, particularly 90% complementarity, more particularly 95%, 96%, 97%, 98%, 99% or 100% complementarity with the corresponding region.

These terms are commonly used in methods related to loop-mediated isothermal amplification (LAMP) methods, such as those described by Nagamine et al. 2002. Molecular and Cellular Probes 16. 223-229.

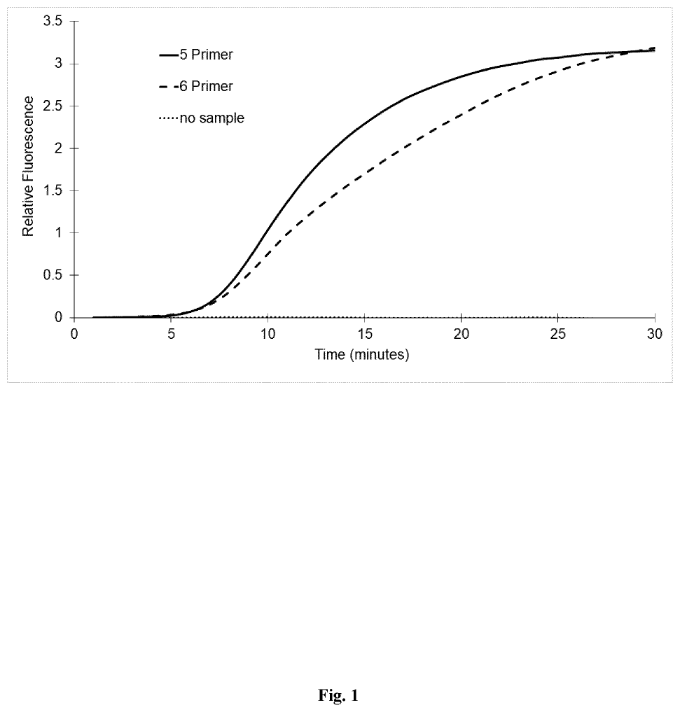

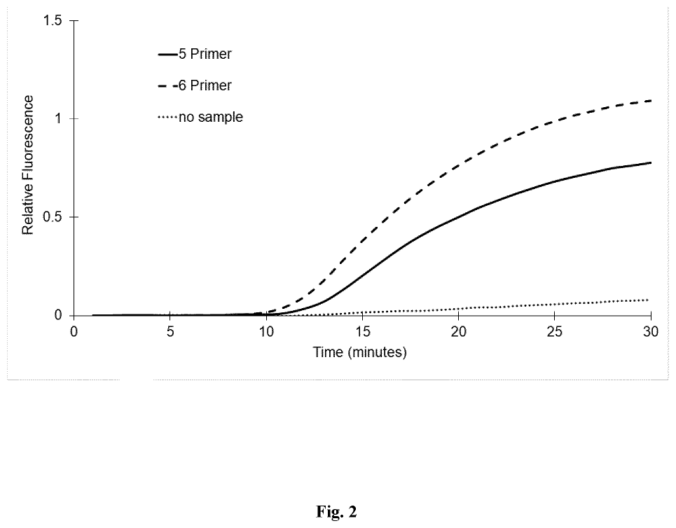

Within the methods of the present invention no F3 primer is used and it is thus preferred that the fifth primer is a B3 primer. This is because it was surprisingly found by the inventors that in the presence of a B3 primer but absence of an F3 primer, detection is as fast and sensitive. Using the methods of the present invention, detection was observed to be possible within ten minutes and as sensitive to detect a low number of pre-determined RNA sequence in a sample (FIG. 1). That is, as it is shown in the appended Examples, a positive detection of a pre-determined RNA sequence of the 16S rRNA of a bacterium of the genus Mycoplasma was achieved using five primers, in particular FIP, BIP, LPF, LPB and B3, within ten minutes after addition of primers and enzymes (FIG. 1). A positive detection of a pre-determined DNA sequence of a Human herpesvirus was achieved using five primers, in particular FIP, BIP, LPF, LPB and B3, within ten minutes after addition of primers and enzymes (FIG. 2). Accordingly, the methods of the present invention, for the first time, provide a reliable and fast way to detect infections which is important in, e.g., controlling a pandemic outbreak of the same.

Within the methods of the present invention, one or more enzyme(s) providing activities of RNA- and/or DNA-dependent DNA polymerase activity and strand-displacement activity are used. That is, in case of an RNA sequence, all three activities are to be added to the RNA sequence to be analyzed. In case of a DNA sequence, activity of the RNA-dependent DNA polymerase is not required. The activities can be provided by one enzyme having all two/three activities, or several enzymes each having one or more of the two/three activities.

It is preferred that the pre-determined nucleic acid sequence is an RNA or DNA sequence.

In some embodiments, it is preferred that the pre-determined RNA or DNA sequence is comprised in a pathogen. That is, the method provided herein is used to detect presence of a nucleic acid sequence of a pathogen in a sample obtained from a human subject. The invention thus relates to, inter alia, a method for diagnosing whether a human subject suffers or is likely to suffer from a disease caused by a pathogen, wherein presence of a nucleic acid sequence of said pathogen was determined using the methods provided herein.

In some embodiments, the pathogen is a virus, a bacterium, a fungus or a parasite.

The term “virus”, as used herein, refers to an infectious agent that replicates only inside the living cells of an organism. Any nucleic acid comprising stadium (e.g. inside the cell or in the virus envelope) may be determined by the invention. In some embodiments, the method of the invention is used to determining the presence of a pre-determined nucleic acid sequence of at least one virus of the genus selected from the group consisting of Adenoviridae, Anelloviridae, Arenaviridae, Astroviridae, Bunyaviridae, Bunyavirus, Caliciviridae, Coronaviridae, Filoviridae, Flaviviridae, Hepadnaviridae, Herpesviridae, Orthomyxoviridae, Papillomaviridae, Paramyxoviridae, Parvoviridae, Picornaviridae, Pneumoviridae, Polyomaviridae, Poxviridae, Reoviridae, Retroviridae, Rhabdoviridae, Rhabdovirus, Togaviridae.

In some embodiments, the method of the invention is used to determining the presence of a pre-determined nucleic acid sequence of at least one bacteria from the genus selected from the group consisting of Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus AlteromonasAmycolata, Amycolatopsis, Anaerobospirillum, Anaerorhabdus, “Anguillina”, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Flexispira, Francisella, Fusobacterium, Gardnerella, Gemella Globicatella, Gordona, Haemophilus, Hafnia, Helicobacter, Helococcus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia and Yokenella.

In some embodiments, the method of the invention is used to determining the presence of a pre-determined nucleic acid sequence of at least one fungus of the genus selected from the group consisting of Candida, Aspergillus, Cryptococcus, Histoplasma, Pneumocystis and/or Stachybotrys.

As used herein, “parasite” refers to an organism that lives in or on a second organism. In some embodiments, the method of the invention is used to determining the presence of a pre-determined nucleic acid sequence of at least one parasite from the genus selected from the group consisting of Ectoparasites, Protozoan organisms and/or Helminths such as Tapeworms, Flukes and/or Roundworms.

It is preferred, in one embodiment, that the primers used in the methods of the invention, in particular for Mycoplasma pneumoniae, are some or preferably all of:

1. FIP primer comprises a sequence of TGC GGG TCC CCG TCA ATT GCC TGG GTA GTA CAT TCG (SEQ ID NO: 1); and/or

2. BIP primer comprises a sequence of CAA GTG GTG GAG CAT GTT TGT CAA GTC TAG GTA AGG (SEQ ID NO: 2), and/or

3. LPF primer comprises a sequence of GTT TGA GTT TCA TTC TTG (SEQ ID NO: 3); and/or

4. LPB primer comprises a sequence of CTT AAT TCG ACG GTA CAC (SEQ ID NO: 4); and/or

5. B3 primer comprises a sequence of TGT TTC CAT AAC TTT GCC (SEQ ID NO: 5).

The above primer sequences target a sequence of Mycoplasma pneumoniae. In particular, the above primer target the following sequence.

#AF132740.1_Mycoplasma_pneumoniae_
strain_ATCC_15531_16S_ribosomal_RNA_gene
and 16S-23S_rRNA_intergenic_spacer_
complete_sequence
(SEQ ID NO: 13)
TTAACGCTGGCGGCATGCCTAATACATGCAAGTCG
ATCGAAAGTAGTAATACTTTAGAGGCGAACGGGTG
AGTAACACGTATCCAATCTACCTTATAATGGGGGA
TAACTAGTTGAAAGACTAGCTAATACCGCATAAGA
ACTTTGGTTCGCATGAATCAAAGTTGAAAGGACCT
GCAAGGGTTCGTTATTTGATGAGGGTGCGCCATAT
CAGCTAGTTGGTGGGGTAACGGCCTACCAAGGCAA
TGACGTGTAGCTATGCTGAGAAGTAGAATAGCCAC
AATGGGACTGAGACACGGCCCATACTCCTACGGGA
GGCAGCAGTAGGGAATTTTTCACAATGAGCGAAAG
CTTGATGGAGCAATGCCGCGTGAACGATGAAGGTC
TTTAAGATTGTAAAGTTCTTTTATTTGGGAAGAAT
GACTTTAGCAGGTAATGGCTAGAGTTTGACTGTAC
CATTTTGAATAAGTGACGACTAACTATGTGCCAGC
AGTCGCGGTAATACATAGGTCGCAAGCGTTATCCG
GATTTATTGGGCGTAAAGCAAGCGCAGGCGGATTG
AAAAGTCTGGTGTTAAAGGCAGCTGCTTAACAGTT
GTATGCATTGGAAACTATTAATCTAGAGTGTGGTA
GGGAGTTTTGGAATTTCATGTGGAGCGGTGAAATG
CGTAGATATATGAAGGAACACCAGTGGCGAAGGCG
AAAACTTAGGCCATTACTGACGCTTAGGCTTGAAA
GTGTGGGGAGCAAATAGGATTAGATACCCTAGTAG
TCCACACCGTAAACGATAGATACTAGCTGTCGGGG
CGATCCCCTCGGTAGTGAAGTTAACACATTAAGTA
TCTCGCCTGGGTAGTACATTCGCAAGAATGAAACT
CAAACGGAATTGACGGGGACCCGCACAAGTGGTGG
AGCATGTTGCTTAATTCGACGGTACACGAAAAACC
TTACCTAGACTTGACATCCTTGGCAAAGTTATGGA
AACATAATGGAGGTTAACCGAGTGACAGGTGGTGC
ATGGTTGTCGTCAGCTCGTGTCGTGAGATGTTGGG
TTAAGTCCCGCAACGAGCGCAACCCTTATCGTTAG
TTACATTGTCTAGCGAGACTGCTAATGCAAATTGG
AGGAAGGAAGGGATGACGTCAAATCATCATGCCCC
TTATGTCTAGGGCTGCAAACGTGCTACAATGGCCA
ATACAAACAGTCGCCAGCTTGTAAAAGTGAGCAAA
TCTGTAAAGTTGGTCTCAGTTCGGATTGAGGGCTG
CAATTCGTCCTCATGAAGTCGGAATCACTAGTAAT
CGCGAATCAGCTATGTCGCGGTGAATACGTTCTCG
GGTCTTGTACACACCGCCCGTCAAACTATGAAAGC
TGGTAATATTTAAAAACGTGTTGCTAACCATTAGG
AAGCGCATGTCAAGGATAGCACCGGTGATTGGAGT
TAAGTCGTAACAAGGTACCCCTACGAGAACGTGGG
GGTGGATCACCTCCTTTCTAATGGAGTTTTTTACT
TTTTCTTTTCATCTTTAATAAAGATAAATACTAAA
CAAAACATCAAAATCCATTTATTTATCGGTGGTAA
ATTAAACCCAAATCCCTGTTTGGTCTCACAACTAA
CATATTTGGTCAGATTGTATCCAGTTCTGAAAGAA
CATTTCCGCTTCTTTCAAAACTGAAAACGACAATC
TTTCTAGTTCCAAATAAATACCAAAGGATCAATAC
AATAAGTTACTAAGGGCTTATGGTG

In some embodiments, the primers used in the methods of the invention, in particular for Mycoplasma pneumoniae, comprise at least one selected from the group of:

a) a FIP primer comprising a sequence that has at least 88%, 91%, 94%, 97% or 100% sequence identity to the sequence: TGC GGG TCC CCG TCA ATT GCC TGG GTA GTA CAT TCG (SEQ ID NO: 1), which sequence still provides the primer functionality,

b) a BIP primer comprising a sequence that has at least 88%, 91%, 94%, 97% or 100% sequence identity to the sequence: CAA GTG GTG GAG CAT GTT TGT CAA GTC TAG GTA AGG (SEQ ID NO: 2), which sequence still provides the primer functionality,

c) a LPF primer comprising a sequence that has at least 88%, 94%, or 100% sequence identity to the sequence: GTT TGA GTT TCA TTC TTG (SEQ ID NO: 3), which sequence still provides the primer functionality,

d) a LPB primer comprising a sequence that has at least 88%, 94%, or 100% sequence identity to the sequence: CTT AAT TCG ACG GTA CAC (SEQ ID NO: 4), which sequence still provides the primer functionality, and

e) a B3 primer comprising a sequence that has at least 88%, 94%, or 100% sequence identity to the sequence: TGT TTC CAT AAC TTT GCC (SEQ ID NO: 5), which sequence still provides the primer functionality, preferably wherein the primer functionality is primer functionality at the SEQ ID NO: 13.

It is preferred, in one embodiment, that the primers used in the methods of the invention, in particular for Human herpesvirus 1, are some or preferably all of:

1. FIP primer comprises a sequence of GTT GGG TGG TGG AGG AGA CGT CCT TTT GGT TCT TGT CGG T (SEQ ID NO: 7); and/or

2. BIP primer comprises a sequence of GGT CGT CCC TCG CAT GAA GCG GCG TGG TAA GGC TGA TG (SEQ ID NO: 8), and/or

3. LPF primer comprises a sequence of TTG GTG GGA ACC CCC GAT AC (SEQ ID NO: 9); and/or

4. LPB primer comprises a sequence of AAC ATG ACC CAG ACC GGC AC (SEQ ID NO: 10); and/or

5. B3 primer comprises a sequence of TAC TTG GCA TGG GGG GTG (SEQ ID NO: 11).

The above primer sequences target a sequence of Human herpesvirus 1. In particular, the above primers target the following sequence.

#AY240834.1_Human herpesvirus_1_isolate_E19_
US4_and_US5_genes_complete_cds_and
US6_gene_partial_cds
(SEQ ID NO: 14)
TGACCGCCCCCGGGGGGCGGTGCTGTTTGCGGGTT
GGCACAAAAAGACCCCGACCCGCGTCTGTGGTGTT
TTTGGCATCATGTCGCCGGGCGCCATGCGTGCCGT
TGTTCCCATTATCCCATTCCTTTTGGTTCTTGTCG
GTGTATCGGGGGTTCCCACCAACGTCTCCTCCACC
ACCCAACCCCAACTCCCGACCACCGGTCGTCCCTC
GCATGAAGCCCCCAACATGACCCAGACCGGCACCA
CCGACTCTCCCACCGCCATCAGCCTTACCACGCCC
GACCACACACCCCCCATGCCAAGTATCGGACTGGA
GGAGGAGGAGGAAGAGGAGGAGGGGGCCGGGGATG
GCGAACATCTTGAGGGGGGAGATGGGACCCGTGAC
ACCCTACCCCAGTCCCCGGGTCCAGCCGTCCCGTT
GGCCGGGGATGACGAGAAGGACAAACCCAACCGTC
CCGTAGTCCCACCCCCCGGTCCCAACAACTCCCCC
GCGCGCCCCGAGACCAGTCGACCGAAGACACCCCC
CACCAGGATCGGGCCGCTGGCAACTCGACCCACGA
CCCAACTCCCCTCAAAGGGGCGACCCTTGGTTCCG
ACGCCTCAACATACCCCGCTGTTCTCGTTCCTCAC
TGCCTCCCCCGCCCTGGACACCCTCTTCGTCATCA
GCACCGTCATCCACACCTTATCGTTTGTGTGTATT
GTTGCGATGGCGACACACCTGTGTGGCGGTTGGTC
CAGACGCGGGCGACGCACACACCCTAGCGTGCGTT
ACGTGTGCCTGCCGCCCGAACGCGGGTAGGGTATG
GGGAGAGCCTACACGCGGAAAGCAAGAACAATAAA
GGCGGCGGGATCTAGTTGATATGCGTCTCTGGGTG
TTTTTGGGGTGTGGCGGGCGCCGGGCGGTCATTGG
ACGGGGTGCAGTTAAATACATGCCCGGGACCCATG
AAGCATGCGCGACTTCCGGGCCTCGGAACCCACCC
GAAACGGCCAACGGACGTCTGAGCCAGGCCTGGCT
ATCCGGAGAAACAACACACGACTTGGCGTTCTGTG
TGTCGCGATGTCTCTGCGCGCAGTCTGGCATCTGG
GGCTTTTGGGAAGCCTCGTGGGGGCTGTTCTTGCC
GCCACCCATCTGGGACCTGCGGCCAACACAACGGA
CCCCTTAACGCACGCCCCAGTGTCCCCTCACCCCA
GCCCCCTGGGGGGCTTTGCCGTCCCCCTCGTAGTC
GGTGGGCTGTGTGCCGTAGTCCTGGGGGCGGCGTG
TCTGCTTGAGCTCCTGCGTCGTACGTGCCGCGGGT
GGGGGCGTTACCATCCCTACATGGACCCAGTTGTC
GTATAAGATGTCGGGTCCAAACTCCCGACACCACC
AGCTGGCATGGTATAAATCACCGGTGCGCCCCCCA
AACCATGTCCGGCAGGGGGATGGGCACCCAACAAC
ACCGGGCTAACCAGGAAATCCGTGGCCCCGGCCCC
CAACAAAGATCGCGGTAGCCCGGCCGTGTGACACT
ATCGTCCATACCGACCACACCGACGAATCCCCTAA
GGGGGAGGGGCCATTTTACGAGGAGGAGGGGTATA
ACAAAGTCTGTCTTTAAAAAGCAGGGGTTAGGGAG
TTGTTCGGTCATAAGCTTCAGCGCGAACGACCAAC
TACCCCGATCATCAGTTATCCTTAAGGTCTCTTTT
GTGTGGTGCGTTCCGGTATGGGGGGGGCTGCCGCC
AGGTTGGGGGCCGTGATTTTGTTTGTCGTCATAGT
GGGCCTCCATGGGGTCCGCGGCAAATATGCCTTGG
CGGATGCCTCTCTT

In some embodiments, the primers used in the methods of the invention, in particular for Human herpesvirus 1, comprise at least one selected from the group of:

a) a FIP primer comprising a sequence that has at least 87%, 90%, 92%, 95%, 97% or 100% sequence identity to the sequence: GTT GGG TGG TGG AGG AGA CGT CCT TTT GGT TCT TGT CGG T (SEQ ID NO: 7), which sequence still provides the primer functionality,

b) a BIP primer comprising a sequence that has at least 89%, 92%, 94%, 97% or 100% sequence identity to the sequence: GGT CGT CCC TCG CAT GAA GCG GCG TGG TAA GGC TGA TG (SEQ ID NO: 8), which sequence still provides the primer functionality,

c) a LPF primer comprising a sequence that has at least 85%, 90%, 95%, or 100% sequence identity to the sequence: TTG GTG GGA ACC CCC GAT AC (SEQ ID NO: 9), which sequence still provides the primer functionality,

d) a LPB primer comprising a sequence that has at least 85%, 90%, 95%, or 100% sequence identity to the sequence: AAC ATG ACC CAG ACC GGC AC (SEQ ID NO: 10), which sequence still provides the primer functionality,

e) a B3 primer comprising a sequence that has at least 88%, 94%, or 100% sequence identity to the sequence: TAC TTG GCA TGG GGG GTG (SEQ ID NO: 11), which sequence still provides the primer functionality,

preferably wherein the primer functionality is primer functionality at the SEQ ID NO: 14.

“Percent (%) sequence identity” with respect to a reference sequence is defined as the percentage of nucleotides in a candidate sequence that are identical with the nucleotides in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

However, the skilled person is well-aware how to design alternative or further primer sequences depending on the target sequence to be detected in the sample (see e.g., Jia, B., et al., 2019, Frontiers in microbiology, 10, 2860).

In the methods of the present invention, in particular in step (c) thereof, the temperature can be fixed.

The term “fixed temperature”, as used herein, refers to keeping the temperature condition constant or almost constant so that enzymes and primers can substantially function. The almost constant temperature condition means that not only the set temperature is accurately maintained but also a slight change in the temperature is acceptable within such a degree that it does not spoil substantial functions of the enzymes and primers. For example, a change in temperature of approximately from 0 to 10° C. is acceptable.

The nucleic acid amplification reaction under a fixed temperature can be carried out by keeping the temperature at such a level that activity of the enzyme to be used can be maintained. In addition, in order to effect annealing of a primer with the target nucleic acid in said nucleic acid amplification reaction, for example, to set the reaction temperature may be set to the temperature of around the Tm value of the primer or lower than that, and it is preferred to set it at a level of stringency by taking the Tm value of the primer into consideration. In said nucleic acid amplification reaction, the amplification reaction can be repeated until the enzyme is inactivated or one of the reagents including primers is used up.

That is, the one or more enzyme(s), DNA primers and the sample to be analyzed are incubated in the same tube at a constant temperature. The temperature is preferably between 50 and 75° C. However, the temperature may also be lower, for example between 30 and 75° C. In an alternative embodiment, a touchdown temperature step is used. That is, the temperature is lowered during the course of the analysis, for example starting at a temperature of 70° C. that is subsequently lowered to 50° C.

In the methods of the present invention, the one or more enzyme(s), DNA primers and the sample to be analyzed are incubated in the same tube for a time between 1 and 120 minutes, preferably between 1 and 60, 1 and 45, 1 and 30 or between 1 and 15 minutes. In a preferred embodiment, the sample is incubated for 3, 4, 5, 6, 7, 8, 9, 10, 11, 12, 13, 14, 15, 16, 17, 18, 19, 20, 21, 22, 23, 24, 25, 26, 27, 28, 29 or 30 minutes.

The methods of the present invention comprise a step of determining whether a double-stranded elongated DNA sequence is present in the sample, in particular wherein presence of the double-stranded elongated DNA sequence in the sample is indicative of the presence of the pre-determined nucleic acid sequence in the sample. The skilled person is well-aware of methods suitable to be used for determining presence of a double-stranded DNA sequence in a sample, in particular where the sequence to be detected is known. Thus, any method known to the skilled person for that purpose may be used within the present invention. However, it is preferred that the presence of the elongated double-stranded DNA is determined by using a nucleic acid molecule hybridisable to the elongated double-stranded DNA sequence, in particular wherein the nucleic acid molecule is labelled, using a molecule that intercalates in the elongated double-stranded DNA sequence or using turbidity measurement.

The term “label” or grammatical variations thereof, as used herein, refer to any detectable or signal-generating molecule or reporter molecule. Convenient labels include colorimetric, chemiluminescent, chromogenic, radioactive and fluorescent labels, but enzymatic (e.g. colorimetric, luminescent, chromogenic) or antibody-based labelling methods or signal-generating systems may also be used. Thus, the term “label” as used herein includes not only directly detectable signal-giving or passive moieties, but also any moiety which generates a signal or takes part in a signal generating reaction or that may be detected indirectly in some way. “labelled” as used herein, refers to being connected with or linked to a detectable label. Determining whether an elongated double-stranded. DNA sequence is present in the sample may be achieved via fluorescence reporting. The majority of such approaches are based on the use of intercalating dyes, such as ethidium bromide, SYBR Green, EvaGreen and YO-PRO-1. (Zhang X, et al. 2013, PLoS One 8(12):e82841: Mair G. et al. 2013, BMC Veterinary Research 9: 108.). As used herein, an agent or dye that “intercalates” refers to an agent or moiety capable of non-covalent insertion between stacked base pairs in a nucleic acid double helix. Determining whether an elongated double-stranded DNA sequence is present in the sample may be achieved by a Fluorescence technique that relies on the mechanism of Forster resonance energy transfer (FRET) (Chen Q, et al., 1997, Biochemistry 36(15):4701-11). In certain embodiments of the invention, the LPB and/or LPF are labelled at the 5′ end with at least one label and/or acceptor fluorophore.

The term “turbidity”, as used herein, refers to a measure of the suspended and/or soluble particles in a fluid or transparent solid that causes light to be scattered or absorbed. In certain embodiments of the invention, indirect determination of whether an elongated double-stranded DNA sequence is present in the sample relies essentially on the formation of pyrophosphate as a reaction byproduct. Pyrophosphate ions can be released by incorporation of deoxynucleotide triphosphates (dNTPs) into the DNA strand during nucleic acid polymerization and these ions react with divalent metal ions, particularly magnesium ions, present in the reaction mix to produce a white, insoluble magnesium pyrophosphate precipitate as described by Mori Y., et al. 2001 (Biochem. Biophys. Res. Commun. 289: 150-154). This participate results in a progressive increase in the turbidity of the reaction solution and pyrophosphate precipitates can be measured quantitatively in terms of turbidity or observed by the naked eye as a pellet after centrifugation. In an alternative embodiment of the invention, determining whether an elongated double-stranded DNA sequence is present in a sample is achieved through the incorporation of manganese ions and calcein in the reaction. Calcein's fluorescence is naturally quenched by binding of manganese ions. Pyrophosphate production as a reaction byproduct removes manganese ions form the buffer through precipitation, and the increased turbidity coupled with restored calcein fluorescence enables an easy visual read-out upon excitation with either visible or UV light (Tomita N., et al. 2008, Nat. Protoc. 3:877-882). In still another embodiment of the invention, the enzymatic conversion of pyrophosphate into ATP, which is produced during DNA synthesis, is monitored through the bioluminescence generated by thermostable firefly luciferase for determining whether an elongated double-stranded DNA sequence is present in the sample (Gandelman OA., et al. 2010, PLoS One 5(11): e14155). Generally, all methods described by Becherer, Lisa, et al. (“Loop-mediated isothermal amplification (LAMP)—review and classification of methods for sequence-specific detection.” Analytical Methods 12.6 (2020): 717-746) can be combined with the method of the invention.

In a further embodiment, the present invention relates to a method of treating a subject infected by a pathogen, the method comprising administering to the subject an efficient amount of a therapeutic drug, wherein the subject has previously been determined to be infected by the pathogen using the method of the present invention.

In a further embodiment, the present invention relates to an anti-infective composition for use in the treatment of an infection of a pathogen, wherein the subject has previously been determined to be infected by the pathogen using the method of the invention.

The term “anti-infective composition”, as used herein, refers to an agent or a composition comprising an antiviral drug, antibiotic drug, antifungal drug and/or antiparasitic drug.

In a preferred embodiment, the pathogen is a virus, a bacterium, a fungus or a parasite. In a further preferred embodiment, the therapeutic drug is an antiviral, antibiotic, antifungal or antiparasitic drug, respectively.

The term “antiviral drug”, as used herein, refers to a drug with properties useful in the treatment against a virus-related disease. An antiviral drug may have, inter alia, properties of preventing, inhibiting, suppressing, reducing, adversely impacting, and/or interfering with the growth, survival, replication, function, and/or dissemination of a virus.

In some embodiments, the antiviral drug described herein comprises at least one agent selected from the group of anti-herpes virus drug, anti-RNA virus drugs and antiretroviral drugs.

In some embodiments, the antiviral drug described herein comprises at least one agent selected from the group of Abacavir, Acyclovir, Adefovir, Amantadine, Ampligen, Amprenavir, Umifenovir, Atazanavir, Atripla, Baloxavir marboxil, Biktarvy, Boceprevir, Bulevirtide, Cidofovir, Cobicistat, Combivir, Daclatasvir, Darunavir, Delavirdine, Descovy, Didanosine, Docosanol, Dolutegravir, Doravirine, Edoxudine, Efavirenz, Elvitegravir, Emtricitabine, Enfuvirtide, Entecavir, Etravirine, Famciclovir, Fomivirsen, Fosamprenavir, Foscarnet, Ganciclovir, Ibacitabine, Ibalizumab, Idoxuridine, Imiquimod, Imunovir, Indinavir, Lamivudine, Letermovir, Lopinavir, Loviride, Maraviroc, Methisazone, Moroxydine, Nelfinavir, Nevirapine, Nexavir, Nitazoxanide, Norvir, Oseltamivir, Penciclovir, Peramivir, Penciclovir, Peramivir, Pleconaril, Podophyllotoxin, Raltegravir, Remdesivir, Ribavirin, Rilpivirine, Rilpivirine, Rimantadine, Ritonavir, Saquinavir, Simeprevir, Sofosbuvir, Stavudine, Taribavirin, Telaprevir, Telbivudine, Tenofovir, Tipranavir, Trifluridine, Trizivir, Tromantadine, Truvada, Umifenovir, Valaciclovir, Valganciclovir, Vicriviroc, Vidarabine, Zalcitabine, Zanamivir and Zidovudine.

The term “antibiotic drug”, as used herein, refers to an agent or a composition with properties useful against bacteria and/or in the treatment of bacteria-related disease. The antibiotic drug may have, inter alia, properties of preventing, inhibiting, suppressing, reducing, adversely impacting, and/or interfering with the growth, survival, replication, function, and/or dissemination of at least one bacterium. In some embodiments, the antibiotic drug described herein comprises at least one agent selected from the group of anti-bacterial phages, macrolides (e.g., erythromycin), penicillins (e.g., nafcillin), cephalosporins (e.g., cefazolin), carbapenems (e.g., imipenem), monobactam (e.g., aztreonam), other beta-lactam antibiotics, beta-lactam inhibitors (e.g., sulbactam), oxalines (e.g. linezolid), aminoglycosides (e.g., gentamicin), chloramphenicol, sufonamides (e.g., sulfamethoxazole), glycopeptides (e.g., vancomycin), quinolones (e.g., ciprofloxacin), tetracyclines (e.g., minocycline), fusidic acid, trimethoprim, metronidazole, clindamycin, mupirocin, rifamycins (e.g., rifampin), streptogramins (e.g., quinupristin and dalfopristin) lipoprotein (e.g., daptomycin) and polyenes (e.g., amphotericin B). In some embodiments, the antibiotic drug described herein comprises at least one agent selected from the group of amoxicillin, azithromycin, amoxicillin/clavulanate, clindamycin, cephalexin, ciprofloxacin, sulfamethoxazole/trimethoprim and metronidazole.

The term “antifungal drug” as used herein, refers to an agent or a composition with properties useful in the treatment against a fungi-related disease. An antifungal drug may have, inter alia, properties of preventing, inhibiting, suppressing, reducing, adversely impacting, and/or interfering with the growth, survival, replication, function, and/or dissemination of at least fungus.

In some embodiments, the antifungal drug described herein comprises at least one agent selected from the group of echinocandins, imidazole antifungals, lanosterol 14α-demethylase inhibitors and triazole antifungals. In some embodiments, the antifungal drug comprises at least one agent selected from the group of Abafungin, Acetic acid, Acrisorcin, Allicin, Aminocandin, Amorolfine, Amphotericin B, Anidulafungin, Bacillomycin, Bifonazole, Blasticidin A, Boric acid, Bromochlorosalicylanilide, Butenafine, Candicidin, Caprylic acid, Caspofungin, Cerulenin, Chlordantoin, Chlormidazole, Chlorophetanol, Chloroxylenol, Ciclopirox, Cilofungin, Cinnamon, Clioquinol, Creolin, Crocodile oil, Cruentaren, Crystal violet, Dimazole, Drosomycin, Echinocandin, Echinocandin B, Ethonam, Fenticlor, Filipin, Griseofulvin, Halicylindramide, Haloprogin, Hamycin, Hinokinin, hydrocortisone, Lufenuron, Luliconazole, Medicinal fungi, Melafix, Micafungin, Miltefosine, Mycobacillin, Natamycin, Nikkomycin, Nystatin, Orotomide, Papulacandin B, Parietin, Pecilocin, Pentamidine, Perimycin, Piroctone olamine, Pneumocandin, Polyene antimycotic, Ptilomycalin A, Pyrrolnitrin, Rimoprogin, Selenium disulfide, Sparassol, Streptomyces isolates, Sulbentine, Tavaborole, Tea tree oil, Terbinafine, Theonellamide F, Thuj aplicin, Thyme, Ticlatone, Tolciclate, Tolnaftate, Trichostatin A, Triclosan, Trimetrexate, Undecylenic acid, Venturicidin, Vinyldithiin, Vusion and Zinc pyrithione.

The term “antiparasitic drug” as used herein, refers to a drug with properties useful in the treatment against a parasite-related disease. An antiparasitic drug may have, inter alia, properties of preventing, inhibiting, suppressing, reducing, adversely impacting, and/or interfering with the growth, survival, replication, function, and/or dissemination of a parasite. In some embodiments, the antiparasitic drug described herein comprises at least one agent selected from the group of abamectin, abametapir, anticestodal agent, arprinocid, arsenamide, ascaricide, avermectin, bephenium hydroxynaphthoate, bithionol, carbadox, clopidol, cymiazole, decoquinate, dichlorophen, diclazuril, diethylcarbamazine, dimetridazole, ectoparasiticide, emodepside, eprinomectin, ethopabate, fexinidazole, flubendazole, halofuginone, hycanthone, isometamidium chloride, ivermectin, lasalocid, malathion, medicinal fungi, melarsomine, metrifonate, milbemycin oxime/lufenuron, narasin, nifurtimox/eflornithine, niridazole, nitazoxanide, nitroxinil, oltipraz, oryzalin, oxamniquine, oxantel, pafuramidine, permethrin, praziquantel, propamidine, quinapyramine, robenidine, salicylhydroxamic acid, salinomycin, selamectin, stibophen, streptomyces isolates, template:anti-arthropod medications, tetraphenylporphine sulfonate, tiabendazole and toltrazuril.

In some embodiments, the invention relates to the anti-infective composition for use of the invention, wherein the pathogen is a Human herpesvirus or a bacterium of the genus Mycoplasma.

The skilled person is aware how to treat an infection with a pathogen once the pathogen has been specified using the methods of the present invention.

The method of the invention can efficiently determine the pathogen and facilitates early detection, screening, monitoring, and/or confirmation of a past infection.

Therefore, the determination of the pathogen enabled by method of the invention can subsequently improve the treatment of an infection, reduce pathogen spreading and/or avoid disease progression.

The invention also relates to a kit, in particular a kit for use in determining presence of a pre-determined nucleic acid sequence in a sample from a human subject. The kit may comprise all of the five primers used in the methods of the present invention or six primers as used herein. The kit may comprise more than one primer system targeting different target sequences, wherein the different target sequences can be part of the same pathogen or different pathogens, e.g. by using primers that contain a quencher-fluorophore duplex region (Tanner N A, Zhang Y, Evans TC Jr. Simultaneous multiple target detection in real-time loop-mediated isothermal amplification. Biotechniques. 2012; 53(2):81-89.). As such, a kit can be used to determine presence of more than one pre-determined nucleic acid sequences in one experiment.

In a particularly preferred embodiment of the present invention, the kits (to be prepared in context) of this invention or the methods and uses of the invention may further comprise or be provided with (an) instruction manual(s). For example, said instruction manual(s) may guide the skilled person (how) to employ the kit of the invention in the diagnostic uses provided herein and in accordance with the present invention. Particularly, said instruction manual(s) may comprise guidance to use or apply the herein provided methods or uses.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

FIG. 1 shows a comparison of the five and six primer system for detecting a pre-determined 16S rRNA sequence in a bacterium of the Mollicutes class using 5 or 6 primers and the methods provided herein.

FIG. 2 shows a comparison of the five and six primer system for detecting a pre-determined DNA sequence in a Human herpesvirus 1 using 5 or 6 primers and the methods provided herein.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

### EXAMPLES

The following are examples of methods and compositions of the invention. It is understood that various other embodiments may be practiced, given the general description provided above.

The Novel 5 Primer System without F3 Amplifies Mollicutes as Efficient as 6 Primer System with F3

TABLE 1
Primers

| | | | |
|---|---|---|---|
| FIP | TGC GGG TCC | LPF | GTT TGA GTT |
| | CCG TCA ATT | | TCA TTC TTG |
| | GCC TGG GTA | | (SEQ ID NO: 3) |
| | GTA CAT TCG | | |
| | (SEQ ID NO: 1) | | |
| BIP | CAA GTG GTG | LPB | CTT AAT TCG |
| | GAG CAT GTT | | ACG GTA CAC |
| | TGT CAA GTC | | (SEQ ID NO: 4) |
| | TAG GTA AGG | | |
| | (SEQ ID NO: 2) | | |
| B3 | TGT TTC CAT | F3 | GTT AAC ACA |
| | AAC TTT GCC | | TTA AGT ATC |
| | (SEQ ID NO: 5) | | (SEQ ID NO: 6) |

TABLE 2
Primer mix: novel 5 primer system

| | Final |
|---|---|
| | concentration. |
| FIP | 1.6 μM |
| BIP | 1.6 μM |
| LPF | 0.8 μM |
| LPB | 0.8 μM |
| B3 | 0.4 μM |

TABLE 3
Primer mix: LAMP 6 primer system

| | Final |
|---|---|
| | concentration |
| FIP | 1.6 μM |
| BIP | 1.6 μM |
| LPF | 0.8 μM |
| LPB | 0.8 μM |
| B3 | 0.2 μM |
| F3 | 0.2 μM |

TABLE 4
Primer/Enzyme mix (PEM)

| | Vol/rx |
|---|---|
| Isothermal master mix | 15.0 μl |
| Primer mix | 2.0 μl |
| | 17.0 μl |

Add 17.0 μl PEM per reaction

Template Addition

Add 8.0 μl extracted RNA

Add 8.0 μl RNase-free H₂O as negative assay control

TABLE 5
Settings for isothermal amplification and dye acquisition

| | Cycles | Temperature | Acquisition | Time | Ramp rate |
|---|---|---|---|---|---|
| Amplification | 25 | 65° C. | None | 27 s | 4.4° C. |
| | | | Single | 30 s | 4.4° C. |
| | | | Quant | Melt | Integration |
| | Channel | Dye | Factor | Factor | Time |
| Dye | #1, | SYBR | 20.00 | 1.2 | Dynamic |
| acquisition | 470/514 | Green I | | | |

The Novel 5 Primer System without F3 Amplifies Human Herpesvirus 1 as Efficient as 6 Primer System with F3

TABLE 6
Primers

| | | | |
|---|---|---|---|
| FIP | GTT GGG TGG | LPF | TTG GTG GGA |
| | TGG AGG AGA | | ACC CCC GAT |
| | CGT CCT TTT | | AC (SEQ ID NO 9) |
| | GGT TCT TGT | | |
| | CGG T | | |
| | (SEQ ID NO: 7) | | |
| BIP | GGT CGT CCC | LPB | AAC ATG ACC |
| | TCG CAT GAA | | CAG ACC |
| | GCG GCG TGG | | GGC AC |
| | TAA GGC TGA TG | | (SEQ ID NO: 10) |
| | (SEQ ID NO: 8) | | |
| B3 | TAC TTG GCA | F3 | GCC GTT GTT |
| | TGG GGG GTG | | CCC ATT ATC |
| | (SEQ ID NO: 11) | | CC (SEQ ID NO: 12) |

TABLE 7
Primer mix: novel 5 primer system

| | Final |
|---|---|
| | concentration. |
| FIP | 1.6 μM |
| BIP | 1.6 μM |
| LPF | 0.8 μM |
| LPB | 0.8 μM |
| B3 | 0.4 μM |

TABLE 8
Primer mix: LAMP 6 primer system

| | Final |
|---|---|
| | concentration |
| FIP | 1.6 μM |
| BIP | 1.6 μM |
| LPF | 0.8 μM |
| LPB | 0.8 μM |
| B3 | 0.2 μM |
| F3 | 0.2 μM |

TABLE 9
Primer/Enzyme mix (PEM)

| | Vol/rx |
|---|---|
| Isothermal master mix | 15.0 μl |
| Primer mix | 2.0 μl |
| | 17.0 μl |

Add 17.0 μl PEM per reaction

Template Addition

Add 8.0 μl extracted DNA

Add 8.0 μl RNase-free H2O as negative assay control

TABLE 10
Settings for isothermal amplification and dye acquisition

| | Cycles | Temperature | Acquisition | Time | Ramp rate |
|---|---|---|---|---|---|
| Amplification | 25 | 65° C. | None | 27 s | 4.4° C. |
| | | | Single | 30 s | 4.4° C. |
| | | | Quant | Melt | Integration |
| | Channel | Dye | Factor | Factor | Time |
| Dye | #1, | SYBR | 20.00 | 1.2 | Dynamic |
| acquisition | 470/514 | Green I | | | |

1. A method for determining presence of a pre-determined nucleic acid sequence in a sample, the method comprising the steps of:
(a) adding one or more enzyme(s) providing activities of RNA- and/or DNA-dependent DNA polymerase activity and strand-displacement activity to the sample to be analysed for the presence of the pre-determined nucleic acid sequence;
(b) adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence;
(c) incubating the sample resulting from steps (a) and (b) at a fixed temperature;
(d) determining whether an elongated DNA sequence is present in the sample, wherein presence of the elongated DNA sequence in the sample is indicative of the presence of the pre-determined nucleic acid sequence in the sample
wherein the sample is obtained from a human subject and wherein no F3 primer is used.

2. The method of claim 1, wherein four of the at least five primers are forward inner primer (FIP), backward inner primer (BIP), loop primer forward (LPF) and loop primer backwards (LPB), respectively.

3. The method of claim 1 or 2, wherein the fifth primer is a B3 primer.

4. The method of any one of claims 1 to 3, wherein the pre-determined nucleic acid sequence is an RNA or DNA sequence.

5. The method of any one of claims 1 to 4, wherein the pre-determined RNA or DNA sequence is comprised in a pathogen.

6. The method of claim 5, wherein the pathogen is a virus, a bacterium, a fungus or a parasite.

7. The method of claim 6, wherein the pathogen is a Human herpesvirus or a bacterium of the genus Mycoplasma.

8. The method of any one of claims 1 to 7, wherein the fixed temperature is between 50 and 75° C.

9. The method of any one of claims 1 to 8, wherein the sample in step (c) is incubated for 1 to 120 minutes.

10. The method of any one of claims 1 to 9, wherein presence of the double-stranded elongated DNA sequence in the sample is determined by using a nucleic acid molecule hybridisable to the double-stranded elongated DNA sequence, in particular wherein the nucleic acid molecule is labelled, using a molecule that intercalates in the double-stranded elongated DNA sequence or using turbidity measurement.

11. An anti-infective composition for use in the treatment of an infection of a pathogen, wherein the subject has previously been determined to be infected by the pathogen using the method of any one of claims 1 to 10.

12. The anti-infective composition for use of claim 11, wherein the pathogen is a virus, a bacterium, a fungus or a parasite.

13. The anti-infective composition for use of claim 11 or 12, wherein the anti-infective composition comprises an antiviral, antibiotic, antifungal or antiparasitic drug, respectively.

14. The anti-infective composition for use of any one of claims 12 to 13, wherein the pathogen is a Human herpesvirus or a bacterium of the genus Mycoplasma.
